(19) United States
(12) Patent Application Publication
(10) Pub. No.: US 2004/0234527 A1
(43) Pub. Date: Nov. 25, 2004

(54) Peptides for use as translocation factors

(76) Inventor: Andrea Crisanti, London (GB)

Correspondence Address: SALIWANCHIK LLOYD & SALIWANCHIK, A PROFESSIONAL ASSOCIATION, PO BOX 142950, GAINESVILLE, FL, 32614-2950, US

(21) Appl. No.: 10/479,166

(22) PCT Filed: Jul. 1, 2002
(86) PCT No.: PCT/GB02/03027

(30) Foreign Application Priority Data
Jun. 29, 2001 (GB) 0116047.2

(51) Int. Cl. A61K 39/395; A61K 38/08; C07K 7/06

(52) U.S. Cl. 424/155.1; 530/388.8; 530/330; 514/17

(57) ABSTRACT

Proteins that contain the amino acid sequence motif X¹X¹X²X³X¹, where X¹=R or K and X² and X³=any amino acid have been found to translocate and can therefore be used in the manufacture of compositions for therapeutic applications. The proteins may also be used as translocation factors to deliver proteins or nucleic acids into a cell.

### FIELD OF THE INVENTION

The present invention relates to the identification of an amino acid motif that aids translocation of proteins, the preparation of proteins as translocation agents and therapeutic agent conjugates.

### BACKGROUND TO THE INVENTION

Gene therapy provides the potential to cure selected genetic diseases. However, a major obstacle is the effective delivery of the gene or protein of interest to the target site. A variety of viral and non-viral vectors have been developed to deliver genes or gene products in to various cells, tissues and organs by ex vivo or in vivo strategies. Among viral-based vectors, retroviruses, adenoviruses, adeno-associated viruses and herpes viruses have been most extensively studied. Among non-viral-based vectors, liposomes and cationic lipid-mediated systems have been used to introduce plasmid DNA directly into animals. However, one of the main challenges of gene therapy remains the design of effective delivery systems.

Histones have also been proposed for use as a vehicle for gene delivery via transfection. Histones are the proteins responsible for the nucleosomal organisation of chromosomes in eukaryotes. The core histones H2A, H2B, H3 and H4 form the core structure of the nucleosome, and the linker histone H1 seals two rounds of DNA at the nucleosomal core.

Zaitsev et al, Gene Therapy, 1997; 4: 586-592 discloses certain nuclear proteins, including histone, which can be prepared to act as DNA carriers via transfection.

Translocation refers to the delivery of proteins across a cell membrane and is therefore different from transfection which relates only to DNA.

Various methods for the delivery of therapeutic proteins across a cell membrane have been proposed. For example, Prochiantz, Current Opinion in Neurobiology, 1996; 6: 629-634, relates to the use of antennapedia to transport proteins across a cell membrane. Other transporting proteins have also been identified including VP22 and tat.

Although these systems are generally useful, it is desirable to identify other suitable transport proteins, particularly those derived from a human source, which would be less likely to be immunogenic.

### SUMMARY OF THE INVENTION

The present invention is based on the surprising finding that proteins and peptides comprising a specific amino acid motif can translocate across a cell membrane.

According to a first aspect of the invention, a protein, or a fragment thereof that retains the biological activity of the protein, comprising an amino acid sequence X¹X¹X²X³X¹, where X¹=R or K and X² and X³=any amino acid, is used in the manufacture of a composition for the treatment of a disease characterised by a deficiency in the production or function of the endogenous protein, or to regulate a biochemical pathway.

In this aspect of the invention, the protein containing the motif can be used in therapy, for example, to replace or supplement a patient's endogenous protein that may be produced inefficiently in the cell. As the protein contains the motif, it will be able to enter the cell by translocation, thereby being delivered to its site of activity. Alternatively, the protein is administered to control a particular biochemical pathway, e.g. a signalling pathway.

According to a second aspect of the invention, a therapeutic agent that has its site of activity within a cell, is used in the manufacture of a composition to treat or diagnose a disease, wherein the agent is conjugated to a peptide that comprises the amino acid sequence defined above. The agent is therefore able to gain entry into the cell using the translocating properties of the peptide.

According to a further aspect of the present invention, there is a conjugate of a peptide capable of translocating across a cell membrane, and a therapeutic, diagnostic or cosmetic agent, wherein the peptide comprises the amino acid sequence motif defined above.

Surprisingly, it has been found that peptides and proteins that comprise the defined amino acid sequence can act via translocation, to deliver a covalently bound therapeutic, diagnostic or cosmetic agent intracellularly.

In contrast to conventional translocating agents such as tat, VP22 or antennapedia, the present invention permits human-derived peptides to be used as the translocation factor, thereby reducing the risk of adverse immunological reactions that may result from the use of non-human peptides.

According to a fourth aspect of the invention, an antibody has affinity for a protein comprising an amino acid sequence as defined above.

According to a fifth aspect of the invention, an expression vector is prepared that expresses a conjugate of the invention in the form of a fusion protein.

### DESCRIPTION OF THE INVENTION

The present invention is based on the surprising finding that peptides comprising a particular amino acid motif are capable of undergoing translocation across a cell membrane. The critical sequence is:

X¹X¹X²X³X¹

where X¹ is Arginine or Lysine and X² and X³ are any amino acid. In the preferred embodiment, X² is Alanine or Valine and X³ is Arginine or Lysine.

Identifying this sequence enables many different translocating peptides to be produced.

In the context of the present invention, the term “translocation” refers to the ability of an agent, protein or conjugate, to cross a cellular membrane, i.e. to enter a cell.

The different motif sequences that may be used in the invention include those shown in Table 1.

TABLE 1

| | | | |
|---|---|---|---|
| KKAKK | KRARK | KRVRK | RRAKK |
| KKAKR | KRAKK | RKAKR | RRAKR |
| KKARK | KRAKR | RKARK | RRARK |
| KKARR | KRARR | RKARR | RRARR |
| KKVKK | KRVKK | RKVKK | RRVKK |
| KKVKR | KRVKR | RKVKR | RRVKR |
| KKVRK | KRVRR | RKVRR | RRVRK |
| KKVRR | KRVRK | RKVRK | RRVRR |

The translocating peptide may be in a truncated form or in a synthetic form, which can be produced readily, without the need to undergo time-consuming and expensive purification steps. Truncated forms are often produced more readily in recombinant expression systems, i.e. in a recombinant mammalian or bacterial expression system. In addition, truncated forms may be less immunogenic and therefore more suitable for administration of the therapeutic agent.

In a preferred embodiment, the translocating peptide fragment comprises no more than 50, preferably no more than 40, and most preferably no more than 30 amino acid residues. The defined sequence motif may be present more than once in the peptide. The peptide is preferably derived from or based on a mammalian protein, preferably a human protein. This reduces the risk of promoting an immunogenic response.

According to the invention, the identification of the defined amino acid motif allows the native protein to be used in therapy. The realisation that a particular endogenous protein has the ability to translocate, enables new therapies to be developed. In this aspect, the natural protein may be administered to a patient, in any pharmaceutically acceptable form, to replace or supplement the endogenous protein, which is either not being produced (or produced in an insufficient amount), or which is produced in an aberrant form. The natural protein is administered knowing that the protein can translocate across the cell membrane, and localise in the defective cells. Accordingly, no other translocation factor is required to facilitate the delivery of the protein.

This therapy is therefore a viable alternative to gene therapy. Rather than attempting to introduce a gene that encodes a product, the product itself can be administered and will translocate across a cellular membrane.

Proteins that comprise the motif may therefore be used in the manufacture of a composition for the treatment of a disease characterised by a deficiency in production of the endogenous protein. Suitable proteins are identified in Tables 2-5, with those proteins shown in Table 2 being of particular interest.

The proteins may not only be administered as a replacement therapy, but may be used in any therapeutic context. For example, the proteins may be required to influence a particular regulatory pathway or to upregulate or downregulate control systems within a cell. Suitable therapies will be apparent to the skilled person.

The proteins to be administered may be produced using techniques known to those skilled in the art. For example, recombinant DNA technology permits the large-scale production of proteins in cell culture, and this can be adapted in the present invention.

Functional variants of the translocating peptides/proteins may also be used. For example, proteins with high levels (greater than 70%, preferably greater than 90% and more preferably greater than 95%) of sequence similarity to the endogenous protein are within the scope of the present invention. The term “similarity” is known in the art. The term refers to a comparison between amino acid sequences, and takes into account not only identical amino acids in corresponding positions, but also functionally similar amino acids in corresponding positions. Thus similarity between polypeptide sequences indicates functional similarity, in addition to sequence similarity.

Levels of similarity between amino acid sequences can be calculated using known methods. In relation to the present invention, publicly available computer based methods for determining similarity include the BLASTP, BLASTN and FASTA programmes (Atschul et al., J. Molec. Biol., 1990; 215:403-410), the BLASTX program available from NCBI, and the Gap program from Genetics Computer Group, Madison Wis. The levels of similarity referred to herein, are determined using the Gap program, with a Gap penalty of 12 and a Gap length penalty of 4.

The variants may be produced using standard recombinant DNA techniques such as site-directed mutagenesis. The variants may also have conserved amino acid substitutions (although not in the critical amino acid sequence), e.g. replacement of a hydrophobic residue for a different hydrophobic residue. All this will be apparent to the skilled person, based on conventional protein technology. The variants must retain the functional ability to translocate across a cellular membrane.

If used as a replacement therapy, to correct a deficiency of the endogenous protein, the variants should also retain the biological activity of the endogenous protein, i.e. that activity that is deficient in the patient.

Compositions containing the protein to be administered may comprise any suitable excipient, diluent or buffer. No other translocating factor is required.

In a preferred embodiment of the invention, the protein to be administered is IRAK1 and/or MYD88. Both of these proteins are involved in the signalling pathway that suppresses production of IL-1 and TNF and these proteins can be translocated into cells to suppress the inflammatory signalling pathway through a decrease in the production of IL-1.

In a further preferred embodiment, the transcription factor E2A is used in therapy. E2A is a molecule known to be involved in controlling proliferation of stem cells, and a mutated form has been identified in childhood leukemia patients. Administering the non-mutated form allows patients to be given a normal source of the protein, helping to ameliorate the condition.

In a further preferred embodiment, Fanconi anaemia can be treated by administering the product of the Fanconi A gene.

In a still further embodiment, Bruton Globulinaemia is treated by administering cytoplasmic tyrosine kinase (NCBI Accession No. Q06187), a mutation in which has been implicated as a causative factor in this disease.

The identification of the motif on particular proteins also enables the identification of suitable therapeutic targets. For example, the realisation that a particular protein has the capability to translocate across a cell membrane, and that in doing so, a disease may be caused or spread, enables the production of suitable therapies designed to prevent this. This is particularly suitable when the protein is a product of an oncogene or a tumourigenic cell. Realising that the protein has the motif and may therefore be implicated in the spread of the disease to neighbouring cells, allows, for example, antibodies to be designed to target the proteins when released from the tumour cell, thereby preventing the proteins from entering other cells.

An example of this is the finding that the protein product of the human APC gene (NCBI Accession No. M74088) contains the motif and that the oncogenic form may be spread by translocation, thereby spreading the disease. The oncogenic product is therefore a viable target for antibody therapy.

Accordingly, the present invention includes antibodies that bind with high affinity to a protein having the defined amino acid motif, and to the use of the antibodies in therapy to treat a disease caused by or promoted by the protein. In particular, the present invention includes antibodies that bind to any of the proteins identified in Tables 2 or 3.

The present invention also includes the inhibition of any of the proteins with the defined motif which are oncogenic, in particular any oncogenic protein of those shown in Tables 2 or 3.

The protein to be administered to a subject may also be used as a cosmetic. For example, DNA-repair proteins that help protect against the damaging effects of UV light or X-ray damage are known. The protein xeroderma pigmentosa, contains the amino acid motif responsible for translocation, and is therefore a suitable product for inclusion in a topical composition for cosmetic use. Used in this way, the protein will translocate across the cell membrane to exert its effect within the target cells. The proteins may therefore be prepared for topical administration, for example in creams or ointments, as will be appreciated by the skilled person.

The present invention also enables peptides comprising the motif to be used as the translocating vehicle for the delivery of other therapeutic, diagnostic or cosmetic agents across a cell membrane to effect entry of the agent into the cell or across an intracellular compartment.

Peptides having the motif may therefore be used to prepare conjugates having the translocating region and a heterologous therapeutic agent.

The term “conjugate” refers to a chimeric molecule formed from a translocating peptide and a therapeutic, diagnostic or cosmetic agent.

The conjugates are therefore hybrid molecules not found together in their natural form. The peptide and agent are covalently linked. The covalent linkage may be in the form of a chemical linker molecule, or the product may be in the form of a fusion protein.

The term “peptide” used herein, is intended to refer to oligo and poly-peptides and proteins.

The translocating peptides may comprise the defined sequence motif more than once, for example two or three motifs may be present.

The translocating peptides may also comprise a high percentage of Lys and Arg residues, typically greater than 5%, preferably more than 10%. The folding of the proteins/peptides and the ionic charge may also be factors that influence translocation.

In addition to the translocating peptides identified herein, the conjugates comprise a discrete, i.e. heterologous, therapeutic, diagnostic or cosmetic agent. In the context of the present invention, a reference to “therapy” or “therapeutic agent” also includes prophylactic treatments, e.g. vaccination. Examples of suitable therapeutic and diagnostic agents include polynucleotides, proteins, peptides, antibodies, enzymes, antigens, growth factors, hormones, non-protein therapeutic or diagnostic agent, enzyme inhibitors, cytotoxic agents and contrast agents.

A protein therapeutic agent is preferably at least 100 amino acids in size. The present invention is particularly useful for longer sequences, e.g. at least 150, 200, 300, 400 or 1000 amino acids in size. For the avoidance of doubt, the term “protein” as used herein also encompasses polypeptides of the required length; although the term “polypeptide” generally means sequences of from 2 to 100 amino acids in length, usually 2 up to 60.

The therapeutic agent may comprise nucleic acid, e.g. a reporter gene. The nucleic acid may be DNA or RNA, in either single stranded or double stranded form.

The nucleic acid may encode a therapeutic agent, e.g. an enzyme, toxin, immunogen, etc. or may itself be the therapeutic agent. For example, anti-sense RNA or DNA may be used to target and disrupt expression of a gene. All this will be apparent to the skilled person.

The therapeutic agent may also be a chemical compound, i.e. an organic or inorganic molecule. Any suitable pharmacological agent is within the scope of the present invention. Preferred chemical molecules include cytoxic agents and growth factors.

It is preferable that the agent to be delivered has its site of activity within a cell. For example, if the agent is a therapeutic protein, the natural site of activity of that protein should be within a cell.

It will be evident to the skilled person which therapeutic agents/proteins have their site of activity within a cell. The agent may be a ligand for a molecule within the cell or may be a target of ligands within the cell. Agents that act outside of the cell are not intended to be within the scope of the invention. For example, agents that act on extracellular receptors, e.g. insulin, do not need to be translocated to exert a therapeutic effect, and are therefore not intended to be a part of the conjugates.

In one embodiment, the conjugates will not contain the sequence KKAKK or KKARK.

The conjugates of the invention may be produced via techniques known to those skilled in the art. The peptide and agent are linked via a covalent attachment. In one embodiment the agent is a peptide (or protein) and the conjugate is a fusion protein. The production of fusion proteins is known to those skilled in the art and comprises the production of a recombinant polynucleotide that encodes, in frame, both the peptide and the agent.

For example, nucleic acid encoding a suitable conjugate may be incorporated into a suitable expression vector or plasmid for further manipulation. As used herein, vector (or plasmid) refers to discrete elements that are used to introduce heterologous DNA into cells for either expression or replication thereof. Selection and use of such vehicles are well known to the skilled person. Many vectors are available, and selection of appropriate vector will depend on the intended use of the vector, e.g. whether it is to be used for DNA amplification or for DNA expression, the size of the DNA to be inserted into the vector, and the host cell to be transformed with the vector. Each vector contains various components depending on its function (amplification of DNA or expression of DNA) and the host cell for which it is compatible. The vector components generally include, but are not limited to, one or more of the following: an origin of replication, one or more marker genes, an enhancer element, a promoter, a transcription termination sequence and a signal sequence.

The conjugates may also be produced by the use of bifunctional reagents which are capable of reacting with the peptide and agent. For example, conjugation of the peptide and agent may be achieved by reagents such as N-succinimidyl 3-(2-pyridyl-dithio)propionate (SPDP) which form a disulphide bridge. Alternative conjugation reagents include: glutaraldehyde, cystamine and EDAC.

Preferably, the agent is linked by a cleavable linker region to the peptide region. Preferably, the cleavable linker region is a protease-cleavable linker, although other linkers, cleavable for example by small molecules, may be used. These include Met-X sites, cleavable by cyanogen bromide, Asn-Gly, cleavable by hydroxylamine, Asp-Pro, cleavable by weak acid, and Trp-X, cleavable by, interalia, NBS-skatole. Protease cleavage sites are preferred due to the milder cleavage conditions necessary and are targeted by, for example, factor Xa, thrombin and collagenase. Any of these may be used. The precise sequences are available in the art and the skilled person will have no difficulty in selecting a suitable cleavage site. By way of example, the protease cleavage region targeted by Factor Xa is I E G R. The protease cleavage region targeted by Enterokinase is D D D D K The protease cleavage region targeted by Thrombin is L V P R G. Preferably, the cleavable linker region is one which is targeted by endocellular proteases.

Additional cell transportation signals may be present. For example, nuclear localisation signals may be an additional component of the constructs.

This may aid the transport of the therapeutic component to the correct intracellular location. Suitable signals are known and identified in the prior art.

The proteins, whether to be administered as a replacement therapy, or for use as a translocating peptide, may also be modified to include additional substituents that help to target the protein/peptide to a particular cell. For example, the proteins may be glycosylated. Alternatively, the proteins/conjugates may be delivered to a particular target tissue/organ by known targetting techniques, for example those based on antibody targetting using liposomes to carry the proteins/conjugates.

It is apparent that the compositions and constructs of the invention are intended for therapeutic use, although diagnostic use is also envisaged. In the context of therapy, it will also be apparent that veterinary use is to be included.

Applications for the conjugates of the present invention include:

1. Antigen Delivery System.

An antigen is any agent that when introduced into an immunocompetent animal stimulates the production of a specific antibody or antibodies that can combine with the antigen. However, the antigen may need to be combined with a carrier to be able to stimulate antibody production or specific T cells (helper or cytotoxic). This is where the present invention may be useful as a carrier for transporting the antigen from one side of the cell membrane to the other such that it can stimulate antibody production. By way of example, bacterial and viral antigens translocated by the conjugates in the cell cytoplasm may be processed and associated with MHC class 1 molecules. This antigen processing and presenting pathway is known to activate specific CD8 cytoxic lymphocytes.

2. Gene Therapy.

Gene therapy may include any one or more of: the addition, the replacement, the deletion, the supplementation, the manipulation etc. of one or more nucleotide sequences in, for example, one or more targeted sites—such as targeted cells. If the targeted sites are targeted cells, then the cells may be part of a tissue or an organ. General teachings on gene therapy may be found in Molecular Biology, Ed Robert Meyers, Pub VCH, such as pages 556-558.

By way of further example, gene therapy can also provide a means by which any one or more of: a nucleotide sequence, such as a gene, can be applied to replace or supplement a defective gene; a pathogenic nucleotide sequence, such as a gene, or expression product thereof can be eliminated; a nucleotide sequence, such as a gene, or expression product thereof, can be added or introduced in order, for example, to create a more favourable phenotype; a nucleotide sequence, such as a gene, or expression product thereof can be added or introduced, for example, for selection purposes (i.e. to select transformed cells and the like over non-transformed cells); cells can be manipulated at the molecular level to treat, cure or prevent disease conditions such as cancer (Schmidt-Wolf and Schmidt-Wolf, 1994, Annals of Hematology 69; 273-279) or other disease conditions, such as immune, cardiovascular, neurological, inflammatory or infectious disorders; antigens can be manipulated and/or introduced to elicit an immune response, such as genetic vaccination. In a particularly preferred embodiment, the compositions may be used to introduce functional proteins in the cytoplasm of genetically deficient cell types.

3. Cancer Therapy.

The conjugates may be used to transport into cancer cells molecules that are transcription factors and are able to restore cell cycle control or induce differentiation. For example, it is understood that many cancer cells would undergo apoptosis if a functional P-53 molecule is introduced into their cytoplasm. The present invention may be used to deliver such gene products. Further, cytotoxic agents may be delivered to the cancer cell to destroy the cell.

4. Antibacterial and Antiviral Therapy.

For example, the compositions may be used to transport in the cytoplasm of infected cells recombinant antibodies or additional DNA-binding molecules which interfere with a crucial step of bacterial and viral replication.

5. Use in Expression Systems for the Production of Protein.

For example, it is desirable to express exogenous proteins in eukaryotic cells in culture so that they get processed correctly. However, many exogenous proteins are toxic to eukaryotic cells. In manufacturing exogenous proteins it is therefore desirable to achieve temporal expression of the exogenous protein. The system may therefore be used in connection with an inducible promoter for this or any other application involving such a system.

6. Contrast Imaging

A suitable contrast agent may be part of the conjugate to allow imaging to be carried out.

7. Cosmetic Methods

The conjugates may also be used to administer cosmetic agents within a cell. For example, the cosmetic agent may be a protein (or nucleic acid encoding a protein) that helps protect against the effects of U.V. damage.

Compositions of the invention may optionally comprise a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may further comprise any suitable binder(s), lubricant(s), suspending agent(s), coating agent(s), solubilising agent(s), and other carrier agents that may aid or increase entry into the target site.

The compositions may be adapted for any route of administration, including intramuscular, intravenous, topical, intradermal or subcutaneous.

The delivery of one or more therapeutic genes or proteins according to the invention may be carried out alone or in combination with other treatments or components of the treatment. Diseases which may be treated include, but are not limited to: cancer, neurological diseases where the agent is required intracellularly, inherited diseases, heart disease, stroke, arthritis, viral infections and diseases of the immune system. Suitable therapeutic genes include those coding for tumour-suppressor proteins, enzymes, pro-drug activating enzymes, immunomodulatory molecules, antibodies, engineered immunoglobulin-like molecules, conjugates, hormones, membrane proteins, vasoactive proteins or peptides, cytokines, chemokines, anti-viral proteins, antisense RNA or DNA and ribozymes.

The amount to be administered to a patient will depend on the usual factors: age of the patient, weight, severity of the condition, route of administration, activity of the therapeutic etc. All this can be determined by conventional methods known to the skilled person.

Proteins that contain the motif include:

1) transcription factors;

2) DNA repair enzymes;

3) proteins involved in the growth and modelling of tissue and bones;

4) oncogenes involved in the genesis; of lung tumour and colon carcinoma; and

5) a series of proteins which have genes that are mutated in a number of heteditary diseases.

Table 2 lists of proteins (or genes for the proteins) that comprise the motif and therefore have the capability to translocate. The proteins may therefore be useful in various aspects of the invention either as a target, e.g. for antibody therapy, for replacement therapy, or as a therapeutic agent for delivery across a cell membrane.

TABLE 2

| Protein/Gene | NCBI Accession No. |
|---|---|
| Viral infectivity factor vif | S43001 |
| Xeroderma pigmentosum group c | AAA82720 |
| Melastatin 1 | NP_002411 |
| Histone deacetylase 1 | NP_004955 |
| Spi-1 proto-oncogene | X52056 |
| Human APC gene | M74088 |
| Homo sapiens coilin (P80) | NM_004645 |
| Bone morphogenetic protein-4 | P12644 |
| Thyrotroph embryonic factor | HSU06935 |
| Bone morphogenetic protein-3 | M22491 |
| dek gene | X64229 |
| Interleukin-1 receptor-associated kinase (IRAK1) | P51617 |
| MYD-88 | AAB49967.1 |
| Fanconi A gene product | 015360 |
| Transcription factor E2A | AAH01728.1 |

Further suitable proteins are listed in Table 3-5, with the NCBI accession numbers identified.

TABLE 3
Search carried out using the ExPASy software with the motif
[R/K]-[R/K]-x-[R/K]-[R/K],
where X = any amino acid.

| | |
|---|---|
| 1. | Neuroendocrine protein 7B2 precursor (Secretory granule endocrine |
| | protein I; Secretogranin V). Acc. No. P05408 |
| 2. | Alzheimer's disease amyloid A4 protein precursor (Protease nexin-II) (PN- |
| | II). Acc No. P05067 |
| 3. | ATP-binding cassette, sub-family A, member 1 (ATP-binding cassette |
| | transporter 1) (ATP-binding cassette 1) (Cholesterol efflux regulatory protein). |
| | Acc. No. 095477 |
| 4. | Proto-oncogene tyrosine-protein kinase ABL1 (p150) (c-ABL). Acc. No. |
| | P00519 |
| 5. | AF-6 protein. Acc. No. P55196 |
| 6. | Autoimmune regulator (APECED protein). Acc. No. O43918 |
| 7. | Amphiregulin precursor (Colorectum cell-derived growth factor) (CRDGF). |
| | Acc. No. P15514 |
| 8. | Apoptotic protease activating factor 1 (Apaf-1). Acc. No. O14727 |
| 9. | Adenomatous polyposis coli protein (APC protein). Acc No. P25054 |
| 10. | Serine-protein kinase ATM (Ataxia telangiectasia mutated) (A-T, mutated). |
| | Acc. No. Q13315 |
| 11. | ADAM-TS 9 precursor (A disintegrin and metalloproteinase with |
| | thrombospondin motifs 9) (ADAMTS-9) (ADAM-TS9). Acc. No. Q9P2N4 |
| 12. | Brain-specific angiogenesis inhibitor 2 precursor (isoform β). Acc. No. |
| | 060241 |
| 13. | Myc box dependent interacting protein 1 (Bridging integrator 1) |
| | (Amphiphysin-like protein) (Amphiphysin II) (Box-dependent myc-interacting |
| | protein-1). Acc No. 000499 |
| 14. | Baculoviral IAP repeat-containing protein 5 (Apoptosis inhibitor survivin) |
| | (Apoptosis inhibitor 4). Acc. No. 015392 |
| 15. | Bloom's syndrome protein. Acc. No. P54132 |
| 16. | Bone morphogenetic protein 3b precursor (Growth/differentiation factor |
| | 10) (GDF-10) (Bone inducing protein). Acc. No. P55107 |
| 17. | Bone morphogenetic protein 2 precursor. Acc. No. P12643 |
| 18. | Bone morphogenetic protein 3 precursor (Osteogenin). Acc. No. P12645 |
| 19. | Bone morphogenetic protein 4 precursor. Acc. No. P12644 |
| 20. | BN51 protein. Acc. No. P05423 |
| 21. | Breast cancer type 1 susceptibility protein. Acc. No. P38398 |
| 22. | Tyrosine-protein kinase BTK (Bruton's tyrosine kinase) |
| | (Agammaglobulinaemia tyrosine kinase) (B cell progenitor kinase). Acc. No. |
| | Q06187 |
| 23. | Brain-cadherin precursor (BR-cadherin) (Cadherin-12) (N-cadherin 2) |
| | (Cadherin, neural type, 2). Acc. No. P55289 |
| 24. | Cadherin-18 precursor (Cadherin-14). Acc. No. Q13634 |
| 25. | Cathepsin L precursor (Major excreted protein). Acc. No. P07711 |
| 26. | Protocadherin alpha 3 precursor (PCDH-alpha3). Acc. No. Q9Y5H8 |
| 27. | Cell division cycle 7-related protein kinase (CDC7-related kinase). Acc. |
| | No. 000311 |
| 28. | Protein kinase CLK2. Acc No. P49760 |
| 29. | Colorectal mutant cancer protein (MCC protein). Acc. No. P23508 |
| 30. | Connective tissue growth factor precursor (Hypertrophic chondrocyte- |
| | specific protein 24). Acc. No. P29279 |
| 31. | Cylicin I (Multiple-band polypeptide I). Acc. No. P35663 |
| 32. | Cylicin II (Multiple-band polypeptide II). Acc. No. Q14093 |
| 33. | Dystroglycan precursor (Dystrophin-associated glycoprotein 1) [Contains: |
| | Alpha-dystroglycan; Beta-dystroglycan. Acc. No. Q14118 |
| 34. | Serine/threonine-protein kinase DCAMKL1 (Doublecortin-like and CAM |
| | kinase-like 1). Acc. No. O15075 |
| 35. | Doublecortin (Lissencephalin-X) (Doublin). Acc. No. 043602 |
| 36. | DEK protein. Acc. No. P35659 |
| 37. | Dyskerin (Nucleolar protein NAP57) (CBF5 homolog). Acc. No. 060832 |
| 38. | Dystrophin. Acc. No. P11532 |
| 39. | Ectodysplasin A (Ectodermal dysplasia protein) (EDA protein). Acc. No. |
| | Q92838 |
| 40. | Steroid hormone receptor ERR1 (Estrogen-related receptor, alpha) (ERR- |
| | alpha) (Estrogen receptor-like 1). Acc. No. P11474 |
| 41. | Steroid hormone receptor ERR2 (Estrogen-related receptor, beta) (ERR- |
| | beta) (Estrogen receptor-like 2) (ERR beta-2). Acc. No. 095718 |
| 42. | Estrogen-related receptor gamma (ERR gamma-2). Acc. No. 075454 |
| 43. | Estrogen receptor (Estradiol receptor) (ER-alpha). Acc. No. P03372 |
| 44. | Endothelin-1 precursor. Acc. No. P05305 |
| 45. | Endothelin-2 precursor. Acc. No. P20800 |
| 46. | Ellis-van Creveld syndrome protein (DWF-1). Acc. No. P57679 |
| 47. | 51 kDa FK506-binding protein (FKBP51) (Peptidyl-prolyl cis-trans |
| | isomerase) (PPiase) (Rotamase) (54 kDa progesterone receptor-associated |
| | immunophilin) (FKBP54) (P54) (FF1 antigen) (HSP90-binding immunophilin). |
| | Acc No. Q13451 |
| 48. | FOSB protein (G0/G1 switch regulatory protein 3). Acc. No. P53539 |
| 49. | P55-C-FOS proto-oncogene protein (Cellular oncogene C-FOS) (G0S7 |
| | protein). Acc. No. P01100 |
| 50. | FOS-related antigen 1. Acc. No. P15407 |
| 51. | FOS-related antigen 2. Acc. No. P15408 |
| 52. | Fragile X mental retardation syndrome related protein 2. Acc. No. P51116 |
| 53. | Growth-arrest-specific protein 7. Acc. No. 060861 |
| 54. | Glucocorticoid receptor. Acc. No. P04150 |
| 55. | Growth/differentiation factor 5 precursor (Cartilage-derived morphogenetic |
| | protein 1). Acc. No. P43026 |
| 56. | Growth/differentiation factor 8 precursor (Myostatin). Acc. No. O14793 |
| 57. | Survival of motor neuron protein-interacting protein 1 (SMN-interacting |
| | protein 1) (Gemin2). Acc. No. 014893 |
| 58. | Granzyme B precursor (T-cell serine protease 1-3E) (Cytotoxic T- |
| | lymphocyte proteinase 2) (Lymphocyte protease) (SECT) (Granzyme 2) |
| | (Cathepsin G-like 1) (Fragmentin 2) (Human lymphocyte protein) (HLP) (C11). |
| | Acc. No. P10144 |
| 59. | Histone H1′ (H1.0) (H1(0)). Acc. No. P07305 |
| 60. | Histone H1A (H1.1). Acc. No. P16401 |
| 61. | Histone H1B (H1.4). Acc. No. P10412 |
| 62. | Histone H1C (H1.3). Acc. No. P16402 |
| 63. | Histone H1D (H1.2). Acc. No. P16403 |
| 64. | Histone H1T. Acc. No. P22492 |
| 65. | Histone H1X. Acc. No. Q92522 |
| 66. | Hypothetical 12.7 kDa histone H2A related protein. Acc. No. P98176 |
| 67. | Core histone macro-H2A.2 (Histone macroH2A2) (mH2A2). Acc. No. |
| | Q9P0M6 |
| 68. | Core histone macro-H2A.1 (Histone macroH2A1) (mH2A1) (H2A.y) |
| | (H2A/y). Acc. No. O75367 |
| 69. | Histone H2B.a/g/k (H2B. 1 A) (H2B/a) (H2B/g) (H2B/k). Acc. No. P02278 |
| 70. | Histone H2B.c (H2B/c). Acc. No. Q99880 |
| 71. | Histone H2B.d (H2B/d). Acc. No. Q99877 |
| 72. | Histone H2B.e (H2B/e). Acc. No. Q99879 |
| 73. | Histone H2B.f (H2B/f) (H2B.1). Acc. No. P33778 |
| 74. | Histone H2B.h (H2B/h). Acc. No. Q93078 |
| 75. | Histone H2B.j (H2B/j). Acc. No. Q93079 |
| 76. | Histone H2B.l (H2B/l). Acc. No. Q93080 |
| 77. | Histone H2B.n (H2B/n) (H2B.2). Acc. No. P23527 |
| 78. | Histone H2B.q (H2B/q) (H2B-GL105). Acc. No. Q16778 |
| 79. | Histone H2B.r (H2B/r) (H2B.1). Acc. No. P06899 |
| 80. | Histone H2B.s (H2B/s). Acc. No. P57053 |
| 81. | Histone H4. Acc. No. P02304 |
| 82. | Heart- and neural crest derivatives-expressed protein 1 (Extraembryonic |
| | tissues, heart, autonomic nervous system and neural crest derivatives-expressed |
| | protein 1). Acc. No. O96004 |
| 83. | Heart- and neural crest derivatives-expressed protein 2. Acc. No. O95300 |
| 84. | Histone deacetylase 1. Acc. No. Q13547 |
| 85. | Antimicrobial peptide hepcidin precursor (Liver-expressed antimicrobial |
| | peptide) (LEAP-1) [Contains: Hepcidin 25; Hepcidin 20]. Acc. No. P81172 |
| 86. | Hepatocellular carcinoma protein HHCM. Acc. No. Q05877 |
| 87. | Hepatic leukemia factor. Acc. No. Q16534 |
| 88. | Heat shock-related 70 kDa protein 2. Acc. No. P54652 |
| 89. | Heat shock cognate 71 kDa protein. Acc. No. P11142 |
| 90. | Heat shock 70 kDa protein 1-HOM. Acc. No. P34931 |
| 91. | Human T-cell leukemia virus enhancer factor. Acc. No. P32314 |
| 92. | Calpain inhibitor (Calpastatin) (Sperm BS-17 component). Acc. No. |
| | P20810 |
| 93. | Caspase-1 inhibitor Iceberg. Acc. No. P57730 |
| 94. | Importin alpha-1 subunit (Karyopherin alpha-1 subunit) (SRP1-beta) (RAG |
| | cohort protein 2) (Nucleoprotein interactor 1) (NPI-1). Acc. No. P52294 |
| 95. | Interleukin-1 receptor-associated kinase 1 (IRAK-1). Acc. No. P51617 |
| 96. | Interferon regulatory factor 2 (IRF-2). Acc. No. P14316 |
| 97. | Kallmann syndrome protein precursor (Adhesion molecule-like X-linked). |
| | Acc. No. P23352 |
| 98. | Nuclear factor NF-kappa-B p100 subunit (H2TF1) (Oncogene LYT-10) |
| | (LYT10). Acc. No. Q00653 |
| 99. | Nuclear factor NF-kappa-B p49 subunit. Acc. No. Q04860 |
| 100. | Serine/threonine-protein kinase PCTAIRE-2. Acc. No. Q00537 |
| 101. | Leukemia associated protein 2. Acc. No. O43262 |
| 102. | Lysosomal trafficking regulator (Beige homolog). Acc. No. Q99698 |
| 103. | Mitogen-activated protein kinase kinase kinase 14 (NF-kappa beta- |
| | inducing kinase) (Serine/threonine protein kinase NIK). Acc. No. Q99558 |
| 104. | Methyl-CpG-binding protein 2 (MeCP-2 protein). Acc. No. P51608 |
| 105. | Thimet oligopeptidase (Endopeptidase 24.15). Acc. No. P52888 |
| 106. | Melanoma-associated antigen D1 (MAGE-D1 antigen). Acc. No. Q9Y5V3 |
| 107. | Melanoma-associated antigen H1 (MAGE-H1 antigen). Acc. No. Q9H213 |
| 108. | Matrix metalloproteinase-15 precursor (MMP-15) (Membrane-type matrix |
| | metalloproteinase 2) (MT-MMP 2) (MTMMP2) (Membrane-type-2 matrix |
| | metalloproteinase) (MT2-MMP) (MT2MMP) (SMCP-2). Acc. No. P51511 |
| 109. | Monocytic leukemia zinc finger protein (Zinc finger protein 220). Acc. No. |
| | Q92794 |
| 110. | Metastasis-associated protein MTA1. Acc. No. Q13330 |
| 111. | MYB-related protein A (A-MYB). Acc. No. P10243 |
| 112. | Myb-related protein B (B-Myb). Acc. No. P10244 |
| 113. | Myogenic factor MYF-5. Acc. No. P13349 |
| 114. | Myogenic factor MYF-6. Acc. No. P23409 |
| 115. | Myogenin (Myogenic factor MYF-4). Acc. No. P15173 |
| 116. | BCL2/adenovirus E1B 19-kDa protein-interacting protein 2. Acc. No. |
| | Q12982 |
| 117. | NK-tumor recognition protein (Natural-killer cells cyclophilin-related |
| | protein) (NK-TR protein). Acc. No. P30414 |
| 118. | Tumor suppressor P53-binding protein 1 (P53-binding protein 1). Acc. |
| | No. Q12888 |
| 119. | Proprotein convertase subtilisin/kexin type 7 precursor (EC 3.4.21.-) |
| | (Proprotein convertase PC7) (Subtilisin/kexin-like protease PC7) (Prohormone |
| | convertase PC7) (Lymphoma proprotein convertase). Acc. No. Q16549 |
| 120. | Polycystin precursor (Autosomal dominant polycystic kidney disease |
| | protein. 1). Acc. No. P98161 |
| 121. | Polycystin 2 (Autosomal dominant polycystic kidney disease type II |
| | protein) (Polycystwin) (R48321). Acc. No. Q13563 |
| 122. | Polymyositis/scleroderma autoantigen 1 (Autoantigen PM/Scl 1) |
| | (Polymyositis/scleroderma autoantigen 75 kDa) (PM/Scl-75) (P75 polymyositis- |
| | scleroderma overlap syndrome associated autoantigen). Acc. No. Q06265 |
| 123. | Prostatic acid phosphatase precursor. Acc. No. P15309 |
| 124. | Pleiotrophin precursor (PTN) (Heparin-binding growth-associated |
| | molecule) (HB-GAM) (Heparin-binding growth factor 8) (HBGF-8) (Osteoblast |
| | specific factor 1) (OSF-1) (Heparin-binding neurite outgrowth promoting factor |
| | 1) (HBNF-1). Acc. No. P21246 |
| 125. | Pituitary tumor-transforming gene 1 protein-interacting protein (Pituitary |
| | tumor-transforming gene protein binding factor) (PTTG-binding factor). Acc. No. |
| | P53801 |
| 126. | 31 kDa transforming protein (Transcription factor PU. 1). Acc. No. P17947 |
| 127. | V(D)J recombination activating protein 1 (RAG-1). Acc. No. P15918 |
| 128. | Retinoblastoma binding protein 1 (RBBP-1). Acc. No. P29374 |
| 129. | Retinoblastoma binding protein 2 (RBBP-2). Acc. No. P29375 |
| 130. | Retinoblastoma-like protein 1 (107 kDa retinoblastoma-associated |
| | protein) (PRB1) (P107). Acc. No. P28749 |
| 131. | Retinoblastoma-like protein 2 (130 kDa retinoblastoma-associated |
| | protein) (PRB2) (P130) (RBR-2). Acc. No. Q08999 |
| 132. | Transforming protein RhoC (H9). Acc. No. P08134 |
| 133. | X-linked retinitis pigmentosa GTPase regulator. Acc. No. Q92834 |
| 134. | Semaphorin 3A precursor (Semaphorin III) (Sema III). Acc. No. Q14563 |
| 135. | Semaphorin 3B precursor (Semaphorin V) (Sema V). Acc. No. Q13214 |
| 136. | Semaphorin 3C precursor (Semaphorin E) (Sema E). Acc. No. Q99985 |
| 137. | Semaphorin 3D precursor. Acc. No. 095025 |
| 138. | Mothers against decapentaplegic homolog 1 (SMAD 1) (Mad-related |
| | protein 1) (Transforming growth factor-beta signaling protein-1) (BSP-1) |
| | (hSMAD1) (JV4-1). Acc. No. Q15797 |
| 139. | Extracellular superoxide dismutase [Cu—Zn] precursor (EC-SOD). Acc. |
| | No. P08294 |
| 140. | Striatin. Acc. No. O43815 |
| 141. | Treacle protein (Treacher collins syndrome protein). Acc. No. Q13428 |
| 142. | Thyrotroph embryonic factor. Acc. No. Q10587 |
| 143. | T-lymphoma invasion and metastasis inducing protein 1 (TIAM1 protein). |
| | Acc. No. Q13009 |
| 144. | TNG1 protein. Acc. No. P56846 |
| 145. | Tumor necrosis factor, alpha-induced protein 2 (B94 protein). Acc. No. |
| | Q03169 |
| 146. | Tuberin (Tuberous sclerosis 2 protein). Acc. No. P49815 |
| 147. | Tetratricopeptide repeat protein 3 (TPR repeat protein D). Acc. No. |
| | P53804 |
| 148. | Utrophin (Dystrophin-related protein 1) (DRP1) (DRP). Acc. No. P46939 |
| 149. | Wiskott-Aldrich syndrome protein interacting protein (WASP interacting |
| | protein) (PRPL-2 protein). Acc. No. O43516 |
| 150. | Wiskott-Aldrich syndrome protein family member 1 (WASP-family protein |
| | member 1) (Verprolin homology domain-containing protein 1). Acc. No. Q92558 |
| 151. | Wiskott-Aldrich syndrome protein family member 2 (WASP-family protein |
| | member 2) (Verprolin homology domain-containing protein 2). Acc. No. |
| | Q9Y6W5 |
| 152. | Wiskott-Aldrich syndrome protein family member 3 (WASP-family protein |
| | members) (Verprolin homology domain-containing protein 3). Acc. No. Q9UPY6 |
| 153. | Neural Wiskott-Aldrich syndrome protein (N-WASP). Acc. No. O00401 |
| 154. | DMA-repair protein complementing XP-B cells (Xeroderma pigmentosum |
| | group B complementing protein) (DNA excision repair protein ERCC-3) (Basal |
| | transcription factor 2 89 kDa subunit) (BTF2-P89) (TFIIH 89 kDa subunit). Acc. |
| | No. P19447 |
| 155. | DNA-repair protein complementing XP-C cells (Xeroderma pigmentosum |
| | group C complementing protein) (P125). Acc. No. Q01831 |
| 156. | DNA-repair protein complementing XP-F cell (Xeroderma pigmentosum |
| | group F complementing protein) (DNA excision repair protein ERCC-4). Acc. |
| | No. Q92889 |
| 157. | DNA-repair protein complementing XP-G cells (Xeroderma pigmentosum |
| | group G complementing protein) (DNA excision repair protein ERCC-5). Acc. |
| | No. P28715 |

TABLE 4
Search carried out using the ExPASy software with the motif
[R/K]-x-[R/K]-[R/K]-[R/K],
wherein X = any amino acid.

| | |
|---|---|
| 1. | Amyloid beta A4 precursor protein-binding family A member 1 (Neuron- |
| | specific X11 protein) (Neuronal Munc18-1-interacting protein 1) (Mint-1) (Adapter |
| | protein X11alpha). Acc. No. Q02410 |
| 2. | Amyloid beta A4 precursor protein-binding family A member 2 (Neuron- |
| | specific X11L protein) (Neuronal Munc18-1-interacting protein 2) (Mint-2) |
| | (Adapter protein X11beta). Acc. No. Q99767 |
| 3. | Apoptosis related protein APR-2. Acc. No. Q9Y5M1 |
| 4. | Beta-secretase precursor (Beta-site APP cleaving enzyme) (Beta-site |
| | amyloid precursor protein cleaving enzyme) (Aspartyl protease 2) (Asp 2) |
| | (ASP2) (Membrane-associated aspartic protease 2) (Memapsin-2). Acc. No. |
| | P56817 |
| 5. | Breast cancer type 2 susceptibility protein. Acc. No. P51587 |
| 6. | Fetal alzheimer antigen (Fetal Alz-50-reactive clone 1). Acc. No. Q12830 |
| 7. | Caspase-10 precursor (ICE-like apoptotic protease 4) (Apoptotic protease |
| | Mch-4) (FAS-associated death domain protein interleukin-1B-converting enzyme |
| | 2) (FLICE2). Acc. No. Q92851 |
| 8. | Insulin-like growth factor IB precursor (IGF-IB) (Somatomedin C). Acc. |
| | No. P05019 |
| 9. | Inhibitor of nuclear factor kappa-B kinase alpha subunit (I kappa-B kinase |
| | alpha) (IkBKA) (IKK-alpha) (IKK-A) (IkappaB kinase) (I-kappa-B kinase 1) (IKK1) |
| | (Conserved helix-loop-helix ubiquitous kinase) (Nuclear factor NFkappaB |
| | inhibitor kinase alpha) (NFKBIKA). Acc. No. O15111 |
| 10. | Interferon alpha-1/13 precursor (Interferon alpha-D) (LeIF D). Acc. No. |
| | P01562 |
| 11. | Tyrosine-protein kinase JAK1 (Janus kinase 1) (JAK-1). Acc. No. P23458 |
| 12. | Melanoma antigen preferentially expressed IN tumors (Preferentially |
| | expressed antigen of melanoma) (OPA-interacting protein 4) (OIP4). Acc. No. |
| | P78395 |
| 13. | Melanoma-associated antigen B1 (MAGE-B1 antigen) (MAGE-XP antigen) |
| | (DSS-AHC critical interval MAGE superfamily 10) (DAM10). Acc. No. P43366 |
| 14. | Myoblast determination protein 1 (Myogenic factor MYF-3). Acc. No. |
| | P15172 |
| 15. | Neurogenic differentiation factor 1 (NeuroD). Acc. No. Q13562 |
| 16. | Neurogenic differentiation factor 2. Acc. No. Q15784 |
| 17. | Neurogenic differentiation factor 3 (Neurogenic basic-helix-loop-helix |
| | protein) (Neurogenin 1). Acc. No. Q92886 |
| 18. | Parathyroid hormone precursor (Parathyrin) (PTH) (Parathormone). Acc. |
| | No. P01270 |
| 19. | RNA-binding protein 5 (RNA binding motif protein 5) (Putative tumor |
| | suppressor LUCA15). Acc. No. P52756 |
| 20. | Ski oncogene (C-ski). Acc. No. P12755 |
| 21. | Spastin. Acc. No. Q9UBP0 |
| 22. | Sex-determining region Y protein (Testis-determining factor). Acc. No. |
| | Q05066 |
| 23. | T-cell acute lymphocytic leukemia-1 protein (TAL-1 protein) (STEM cell |
| | protein) (T-cell leukemia/lymphoma-5 protein). Acc. No. P17542 |
| 24. | Tumor necrosis factor ligand superfamily member 13 (A proliferation- |
| | inducing ligand) (APRIL) (TNF-and APOL-related leukocyte expressed ligand 2) |
| | (TALL-2) (TNF-related death ligand-1) (TRDL-1). Acc. No. 075888 |
| 25. | Tumor necrosis factor receptor superfamily member 18 precursor |
| | (Glucocorticoid-induced TNFR-related protein) (Activation-inducible TNFR |
| | family receptor). Acc. No. Q9Y5U5 |
| 26. | Trichohyalin. Acc. No. Q07283 |
| 27. | Vascular endothelial growth factor B precursor (VEGF-B) (VEGF related |
| | factor). Acc. No. P49765 |
| 28. | Vascular endothelial growth factor precursor (VEGF) (Vascular |
| | permeability factor) (VPF). Acc. No. P15692 |

TABLE 5
Search carried out using the ExPASy software
with the motif [R/K]-[R/K]-[R/K]-x-[R/K],
wherein X = any amino acid.

| | |
|---|---|
| 1. | Transcription factor AP-1 (Proto-oncogene C-JUN) (P39) |
| | (G0S7). Acc. No. P05412 |
| 2. | T-cell surface glycoprotein CD3 zeta chain precursor |
| | (T-cell receptor T3 zeta chain). Acc. No. P20963 |
| 3. | T lymphocyte activation antigenCD86 precursor (Activation |
| | B7-2 antigen) (CTLA-4 counter-receptor B7.2) (FUN-1). |
| | Acc. No. P42081 |
| 4. | T-cell surface glycoprotein CD8 beta chain precursor |
| | (Antigen CD8B). Acc. No. P10966 |
| 5. | Cell division cycle 2-like protein kinase 5 |
| | (Cholinesterase-related cell division controller) |
| | (CDC2-related protein kinase 5). Acc. No. Q14004 |
| 6. | Melanoma-associated antigen B2 (MAGE-B2 antigen) |
| | (DSS-AHC critical interval MAGE superfamily 6) |
| | (DAM6). Acc. No. 015479 |
| 7. | Parathyroid hormone-related protein precursor (PTH-rP) |
| | (PTHrP). Acc. No. P12272 |
| 8. | Major prion protein precursor (PrP) (PrP27-30) |
| | (PrP33-35C) (ASCR). Acc. No. P04156 |
| 9. | Transforming growth factor beta 2 precursor (TGF-beta 2) |
| | (Glioblastoma-derived T-cell suppressor factor) (G-TSF) |
| | (BSC-1 cell growth inhibitor) (Polyergin) (Cetermin). |
| | Acc. No. P08112 |
| 10. | Transformer-2 protein homolog (TRA-2 alpha). Acc. |
| | No. Q13595 |
| 11. | DNA-repair protein complementing XP-B cells (Xeroderma |
| | pigmentosum group B complementing protein) (DNA excision |
| | repair protein ERCC-3) (Basal transcription factor 2 89 |
| | kDa subunit) (BTF2-P89) (TFIIH 89 kDa subunit). Acc. |
| | No. P19447 |
| 12. | DNA-repair protein complementing XP-C cells (Xeroderma |
| | pigmentosum group C complementing protein) (P125). |
| | Acc. No. Q01831 |
| 13. | Hypothetical 28.3 kDa protein (TCF3 (E2A) fusion partner |
| | in childhood leukemia). Acc. No. Q9UPA6 |

The following Examples illustrate the invention.

### EXAMPLE 1

This experiment was carried out to compare the translocation efficiency of different protein delivery systems: lipofectin reagent, antennapedia and histone H1.4B. The histone H1.4B protein contained the defined motif.

The quantitative measurement of translocation was carried out by reading the activity of a β-Galactosidase enzyme that was included as part of a fusion protein with the translocation agents. When provided with the appropriate galactoside substrate, the enzyme deglycosylates the substrate leading to the accumulation of dioxetane. During a following incubation in a different buffer, the dioxetane becomes deprotonated and decomposes with emission of light (425 nm) that can be read using a luminometer. This system, therefore, provided a sensitive means of detecting the amount of the β-Galactosidase present in the samples, and ultimately, the amount of fusion protein delivered into the cells.

The experiments were carried out using 8-well chamber slides. 3×10⁴ Hela cells were seeded into each well, suplemented with 400 μl RPMI+10% FCS medium and incubated overnight at 37° C. and 5% CO₂. The following day, the appropriate dilutions of protein were made into RPMI medium and added to the wells. Both lipofectin delivery and translocation experiments were carried out for 4 hours.

The experiments were carried out on two different days: the first day a negative control β-Galactosidase and the H1.4B-Bgal were tested, and the second day the antennapedia fusion (Antp-βgal) was tested. A lipofectin delivery of β-Galactosidase, provided by Roche, was performed on both days as positive control for delivery into the cells.

The amount of each protein construct was determined after previous tests that showed these were the best values to obtain a linear reading using a luminometer. It was decided to start with the amount of each that gave a value close to 6×10⁷ rlu, as this is the limit of the equipment's capacity. From that value, three consecutive ⅕ fold dilutions were made in each case.

These tests also showed that the different constructs containing the β-Galactosidase had different activities. Therefore, the original activity of the different proteins was also measured in parallel to the activity recovered after translocation, in order to make sure that all the proteins were standardised to an original maximum activity of around 6×10⁷ rlu.

The experimental protocol was as follows:

1. Wash cells (split previous night into chamber slides: 2-3×10⁴ cells in 0.4 ml complete RPMI/well) 2 times with incomplete RPMI.

2. Add 0.5 ml complete RPMI/well.

3. Add peptide (from 200 ng/well diluted in steps of 1:2 to blank).

4. Leave for 4 hours in incubator.

Fixing of Chamber Slides and Development with Avidin-Fluorescein

1. Remove medium from chamber slides.

2. Wash slide 3 times with 1×PBS.

3. Eliminate most liquid, pull off chamber and let the slide dry.

4. Freeze at −20° C. for at least 20 min.

5. Fix in 3% formaldehyde in 1×PBS for 15-20 min.

6. Rinse in 0.5% BSA in PBS.

7. Block with 5% BSA in 1×PBS for 20 min.

8. Rinse 3 times with 0.5 M NaCl in PBS.

9. Add Avidin-fluorescein solution (1:4000 dilution in 0.1 M NaHCO₃, 0.5 M NaCl, pH 8.5) 50 μl/well and incubate for 30 min in dark humid chamber.

10. Wash 10 times with 0.5 M NaCl in PBS.

11. Remove plastic sections around wells.

12. Add 2-3 drops of 4,6-diamidino-2-phenylindole (DAPI) or fluorescent stain and seal slide with nail varnish.

13. Check in microscope and store in dark humid chamber.

The results after translocation were obtained by reading the β-Galactosidase recovered from the cell extracts and are shown in Tables 6 and 7. In all cases the values obtained follow a linear pattern which indicates that the system is efficient. The results for the β-Galactosidase negative controls shows an increasing tendancy that does not correlate with original expectations, as this molecule does not translocate and no difference of activity should be observed when the amount of protein is increased. However, this artefact may be due to the washes or to molecules of protein that did not get into the cells but attached to the cellular membrane when they were added to the wells. In any case, the values for the biggest amounts of β-Galactosidase protein are clearly lower than those obtained from the delivery systems.

As for the comparison between lipofectin delivery, Antp-β-Gal and H1.4B-β-Gal, the histone H1.4B translocates at a rate five times higher than that of lipofectin and antennapedia, as the β-Galactosidase activity recovered after translocation is five times higher.

TABLE 6

| | | |
|---|---|---|
| Amount of | Protein activity | Activity after |
| protein | (rlu) | transfection (rlu) |

β-Gal positive control (Roche) transfection

| Blank | | 8.578 | 27.347 |
|---|---|---|---|
| Blank | | 10.204 | 26.662 |
| 1.6 | ng | 22.026.968 | 50.353 |
| 1.6 | ng | 22.101.678 | 47.212 |
| 8 | ng | 43.535.464 | 140.342 |
| 8 | ng | 43.392.116 | 162.319 |
| 40 | ng | 62.436.592 | 635.228 |
| 40 | ng | 61.919.408 | 675.640 |

β-Galactosidase translocation

| | | | |
|---|---|---|---|
| 640 | pg | 6.948.195 | 25.076 |
| 640 | pg | 5.911.789 | 28.710 |
| 3.2 | ng | 51.004.204 | 29.527 |
| 3.2 | ng | 50.660.716 | 35.227 |
| 15 | ng | 24.657.924 | 56.941 |
| 16 | ng | 24.504.798 | 55.164 |
| 80 | ng | overload | 242.200 |
| 80 | ng | overload | 298.589 |

H1.4B-β-Gal translocation

| | | | |
|---|---|---|---|
| 1.6 | ng | 3.929.454 | 49.243 |
| 1.6 | ng | 3.694.802 | 35.667 |
| 8 | ng | 11.583.624 | 70.352 |
| 8 | ng | 12.377.050 | 60.663 |
| 40 | ng | 33.230.640 | 355.641 |
| 40 | ng | 33.458.668 | 333.496 |
| 200 | ng | 58.670.112 | 2.687.583 |
| 200 | ng | 56.713.392 | 2.989.107 |

TABLE 7

| | | |
|---|---|---|
| Amount of | Protein activity | Activity after |
| protein | (rlu) | transfection (rlu) |

β-Gal positive control (Roche) transfection

| Blank | | 564 | 2.417 |
|---|---|---|---|
| Blank | | 503 | 1.835 |
| 1.6 | ng | 10.302.814 | 40.121 |
| 1.6 | ng | 10.740.756 | 33.578 |
| 8 | ng | 33.610.784 | 164.745 |
| 8 | ng | 34.080.292 | 157.913 |
| 40 | ng | 57.920.416 | 715.773 |
| 40 | ng | 57.083.376 | 744.405 |

Antp-β-Gal translocation

| | | | |
|---|---|---|---|
| 2 | ng | 618.178 | 2.275 |
| 2 | ng | 628.733 | 3.020 |
| 10 | ng | 2.638.336 | 2.028 |
| 10 | ng | 2.660.380 | 2.027 |
| 50 | ng | 10.787.237 | 39.725 |
| 50 | ng | 11.323.412 | 25.066 |
| 250 | ng | 36.136.704 | 549.104 |
| 250 | ng | 37.321.488 | 682.181 |

Tat-β-Gal translocation

| | | | |
|---|---|---|---|
| 0.8 | ng | 1.638.342 | 4.317 |
| 0.8 | ng | 1.620.746 | 5.587 |
| 4 | ng | 6.443.625 | 6.318 |
| 4 | ng | 5.424.017 | 5.454 |
| 20 | ng | 20.843.930 | 30.984 |
| 20 | ng | 22.358.902 | 25.516 |
| 100 | ng | 53.224.476 | 220.384 |
| 100 | ng | 49.537.884 | 128.548 |

### EXAMPLE 2

Various peptides were prepared synthetically and conjugated to a biotin molecule. The conjugates were then tested for translocation using the experimental protocol described in Example 1.

The conjugates are identified in Table 8, together with the identification of the proteins from which they are derived.

TABLE 8

| | | | |
|---|---|---|---|
| Protein name | Genbank Nb | Peptide amino acid sequence | SEQ ID NO. |

| | | | |
|---|---|---|---|
| TEF | gi:4507431 | Biotin-MIKKAKKVFVPDEQKDEK-Amide | 1 |
| BMP-3 | gi:115072 | Biotin-TLKKARRKQWIEPRNCARR-Amide | 2 |
| Spi-1 | gi:4507175 | Biotin-GEVKKVKKKLTYQFSGEVL-Amide | 3 |
| APC | gi:182397 | Biotin-SSRKAKKPAQTASKLPPPVAR-Amide | 4 |
| P80-coilin | gi:4758024 | Biotin-NLSLRKAKKRAFQLEEG-Amide | 5 |
| BMP-4 | gi:115073 | Biotin-HALTRRRRAKRSPKHHSQ-Amide | 6 |
| IRAK | gi:8928535 | Biotin-CLHRRAKRRPPMTQVYER-Amide | 7 |
| Melastatin1 (p2) | gi:3243075 | Biotin-TKGGRGKGKGKKKGKVK-Amide | 8 |
| HDAC1 | gi:13128860 | Biotin-SNFKKAKRVKTEDEKEKDP-Amide | 9 |
| Caspase-1 | gi:10954343 | Biotin-QLLRKKRRIFIHSVGAGT-Amide | 10 |
| inhibitor Iceberg | | | |
| Dyskerin p1 | gi:4503337 | Biotin-IKKEKKKSKKDKKAKAGLES-Amide | 11 |
| Dyskerin p2 | gi:4503337 | Biotin-LPKKHKKKKERKSLPEED-Amide | 12 |
| E2A leukemia p1 | gi:12804613 | Biotin-AARGRRRRQRELNRRKYQA-Amide | 13 |
| E2A leukemia p2 | gi:12804613 | Biotin-GPSGRKRRRVPRDGRRAGNA-Amide | 14 |
| Major prion | gi:2944217 | Biotin-GLCKKRPKPGGWNTGGSR-Amide | 15 |
| protein precursor | | | |
| Bruton's tyrosine | gi:2117817 | Biotin-HRKTKKPLPPTPEEDQILKKP-Amide | 16 |
| kinase | | | |
| Sex-determining | gi:2269817 | Biotin-PNYKYRPRRKAKMLPKNCS-Amide | 17 |
| region Y protein | | | |
| Baculoviral IAP repeat- | gi:4502145 | Biotin-AKETNNKKKEFEETAKKVRRA-Amide | 18 |
| Containing protein 5 | | | |
| Apoptosis Inhibitor | gi:2315863 | Biotin-NKIAKETNNKKKEFEETAKKVRRA-Amide | 19 |
| Survivin | | | |
| Granzyme B | gi:4758494 | Biotin-QLERKAKRTRAVQPLRLPS-Amide | 20 |
| Precursor | | | |
| Parathyroid hormone | gi:4506267 | Biotin-SQRPRKKEDNVLVESHEKSLGE-Amide | 21 |
| Toll-interleukin 1 | gi:20140483 | Biotin-GKMADWFRQTLLKKPKKRPNSPEST-Amide | 22 |
| receptor | | | |
| domain-containing | | | |
| adapter protein | | | |

In each case, translocation was observed.

1. (currently cancelled).

2. The method, according to claim 9, wherein X²=A or V.

3. The method, according to claim 9, wherein X³=R or K.

4. The method, according to claim 9, wherein X³=R or K.

5. The method, according to claim 9, wherein the amino acid sequence is KKAKK or KKVKK.

6. The method, according to claim 9, wherein said protein is a human-derived protein.

7. The method, according to claim 9, wherein the protein is any of those identified in Table 2.

8. The method, according to claim 9, wherein the protein is selected from the group consisting of: IRAK1, MYD88, transcription factor E2A, Fanconi A gene product and cytoplasmic tyrosine kinase.

9. A method for the treatment of disease, the disease being characterised by a deficiency in the production or function of an endogenous protein comprising the amino acid sequence X¹X¹X²X³X¹, where X¹=R or K and X² and X³=any amino acid, wherein said method comprises administering to a patient in need of such treatment a composition comprising the functional protein, or a functional fragment thereof comprising the defined amino acid sequence.

10. An antibody having affinity for a protein comprising an amino acid sequence X¹X¹X²X³X¹, where X¹=R or K and X² and X³=any amino acid.

11. The antibody, according to claim 10, wherein the protein is any of those identified in Table 2, or a mutant thereof.

12. The antibody according to claim 10, wherein the protein is a product of an oncogene.

13. The antibody according to claim 10, which has affinity for the protein product of the human APC gene.

14. A conjugate of a peptide capable of translocation across a cell membrane, and a therapeutic or diagnostic agent, wherein the peptide comprises the amino acid sequence X¹X¹X²X³X¹, where X¹ is R or K and X² and X³=any amino acid.

15. The conjugate according to claim 14, wherein X¹=K.

16. The conjugate according to claim 14, wherein X²=A or V.

17. The conjugate according to claim 14, wherein X³=R or K.

18. The conjugate according to claim 16, wherein X³=R or K.

19. The conjugate according to claim 14, with the proviso that the peptide does not contain the sequence KKAKK or KKARK.

20. The conjugate according to claim 14, wherein the peptide comprises the amino acid sequence KKVKK.

21. The conjugate according to claim 14, wherein the agent is a therapeutic protein.

22. The conjugate according to claim 14, wherein the agent is an antibody.

23. The conjugate according to claim 22, wherein the antibody has affinity for a protein comprising the sequence X¹X¹X²X³X¹, where X¹=R or K and X² and X³=any amino acid.

24. The conjugate according to claim 14, wherein the agent has its site of activity within a cell.

25. The conjugate according to claim 14, wherein the agent is a polynucleotide molecule.

26. The conjugate according to claim 14, wherein the agent is a contrast agent.

27. The conjugate according to claim 14, which is a fusion protein.

28. The conjugate according to claim 14, wherein the agent is conjugated to the peptide via a chemical linker molecule.

29. The conjugate according to claim 14, wherein the peptide comprises at least 20 amino acids.

30. A method for therapy or diagnosis wherein said method comprises the use of a conjugate of a peptide capable of translocation across a cell membrane, and a therapeutic or diagnostic agent, wherein the peptide comprises the amino acid sequence X¹X¹X²X³X¹X where X¹ is R or K and X² and X³=any amino acid.

31. An expression vector that encodes a fusion protein wherein said fusion protein is a conjugate of a peptide capable of translocation across a cell membrane, and a therapeutic or diagnostic agent, wherein the peptide comprises the amino acid sequence X¹X¹X²X³X¹, where X¹ is R or K and X² and X³=any amino acid.

32. A recombinant cell line comprising an expression vector that encodes a fusion protein wherein said fusion protein is a conjugate of a peptide capable of translocation across a cell membrane, and a therapeutic or diagnostic agent, wherein the peptide comprises the amino acid sequence X¹X¹X²X³X¹ where X¹ is R or K and X² and X³=any amino acid.

33. A process for the production of a therapeutic or diagnostic agent capable of being delivered across a cell membrane, comprising covalently attaching the agent to a peptide comprising the amino acid sequence X¹X¹X²X³X¹, where X¹=R or K and X² and X³=any amino acid.

34. A method for providing a therapeutic effect within a cell wherein said method comprises contacting the cell with a therapeutic agent wherein the agent is conjugated to a peptide that comprises the amino acid sequence X¹X¹X²X³X¹, where X¹=R or K and X² and X³=any amino acid.

35. A method for the treatment or diagnosis of a disease, wherein said method comprises the use of a conjugate of a peptide capable of translocation across a cell membrane, wherein the peptide comprises the amino acid sequence X¹X¹X²X³X¹, where X¹=R or K and X² and X³=any amino acid.

36. A method for the treatment of a tumour, wherein said method comprises contacting the tumour with an antibody wherein the antibody has affinity for a protein product of an oncogene, and comprises an amino acid sequence X¹X¹X²X³X¹, where X¹=R or K and X² and X³=any amino acid.

37. (currently cancelled).

38. A composition, for therapeutic use, consisting of a protein comprising an amino acid sequence X¹X¹X²X³X¹, where X¹=R or K and X² and X³=any amino acid, and a pharmaceutically acceptable carrier.

39. The composition, according to claim 38, wherein the protein is capable of translocation across a cell membrane and the composition does not comprise any additional translocation factor to facilitate delivery of the protein.


SEQUENCE LISTING (22 sequences)

SEQ ID NO: 1 - protein, 18 aa, Artificial Sequence
  Description of Artificial Sequence Synthetic peptide conjugated with biotin
MIKKAKKVFVPDEQKDEK

SEQ ID NO: 2 - protein, 19 aa, Artificial Sequence
  Description of Artificial Sequence Synthetic peptide conjugated with biotin
TLKKARRKQWIEPRNCARR

SEQ ID NO: 3 - protein, 19 aa, Artificial Sequence
  Description of Artificial Sequence Synthetic peptide conjugated with biotin
GEVKKVKKKLTYQFSGEVL

SEQ ID NO: 4 - protein, 21 aa, Artificial Sequence
  Description of Artificial Sequence Synthetic peptide conjugated with biotin
SSRKAKKPAQTASKLPPPVAR

SEQ ID NO: 5 - protein, 17 aa, Artificial Sequence
  Description of Artificial Sequence Synthetic peptide conjugated with biotin
NLSLRKAKKRAFQLEEG

SEQ ID NO: 6 - protein, 18 aa, Artificial Sequence
  Description of Artificial Sequence Synthetic peptide conjugated with biotin
HALTRRRRAKRSPKHHSQ

SEQ ID NO: 7 - protein, 18 aa, Artificial Sequence
  Description of Artificial Sequence Synthetic peptide conjugated with biotin
CLHRRAKRRPPMTQVYER

SEQ ID NO: 8 - protein, 17 aa, Artificial Sequence
  Description of Artificial Sequence Synthetic peptide conjugated with biotin
TKGGRGKGKGKKKGKVK

SEQ ID NO: 9 - protein, 19 aa, Artificial Sequence
  Description of Artificial Sequence Synthetic peptide conjugated with biotin
SNFKKAKRVKTEDEKEKDP

SEQ ID NO: 10 - protein, 18 aa, Artificial Sequence
  Description of Artificial Sequence Synthetic peptide conjugated with biotin
QLLRKKRRIFIHSVGAGT

SEQ ID NO: 11 - protein, 20 aa, Artificial Sequence
  Description of Artificial Sequence Synthetic peptide conjugated with biotin
IKKEKKKSKKDKKAKAGLES

SEQ ID NO: 12 - protein, 18 aa, Artificial Sequence
  Description of Artificial Sequence Synthetic peptide conjugated with biotin
LPKKHKKKKERKSLPEED

SEQ ID NO: 13 - protein, 19 aa, Artificial Sequence
  Description of Artificial Sequence Synthetic peptide conjugated with biotin
AARGRRRRQRELNRRKYQA

SEQ ID NO: 14 - protein, 20 aa, Artificial Sequence
  Description of Artificial Sequence Synthetic peptide conjugated with biotin
GPSGRKRRRVPRDGRRAGNA

SEQ ID NO: 15 - protein, 18 aa, Artificial Sequence
  Description of Artificial Sequence Synthetic peptide conjugated with biotin
GLCKKRPKPGGWNTGGSR

SEQ ID NO: 16 - protein, 21 aa, Artificial Sequence
  Description of Artificial Sequence Synthetic peptide conjugated with biotin
HRKTKKPLPPTPEEDQILKKP

SEQ ID NO: 17 - protein, 19 aa, Artificial Sequence
  Description of Artificial Sequence Synthetic peptide conjugated with biotin
PNYKYRPRRKAKMLPKNCS

SEQ ID NO: 18 - protein, 21 aa, Artificial Sequence
  Description of Artificial Sequence Synthetic peptide conjugated with biotin
AKETNNKKKEFEETAKKVRRA

SEQ ID NO: 19 - protein, 24 aa, Artificial Sequence
  Description of Artificial Sequence Synthetic peptide conjugated with biotin
NKIAKETNNKKKEFEETAKKVRRA

SEQ ID NO: 20 - protein, 19 aa, Artificial Sequence
  Description of Artificial Sequence Synthetic peptide conjugated with biotin
QLERKAKRTRAVQPLRLPS

SEQ ID NO: 21 - protein, 22 aa, Artificial Sequence
  Description of Artificial Sequence Synthetic peptide conjugated with biotin
SQRPRKKEDNVLVESHEKSLGE

SEQ ID NO: 22 - protein, 25 aa, Artificial Sequence
  Description of Artificial Sequence Synthetic peptide conjugated with biotin
GKMADWFRQTLLKKPKKRPNSPEST